(19) United States
(12) Patent Application Publication
(10) Pub. No.: US 2007/0244377 A1
(43) Pub. Date: Oct. 18, 2007

(54) PULSE OXIMETER SLEEVE

(76) Inventors: Jenny L. Cozad, Pauls Valley, OK (US); Leslie L. Jensen, Elmore City, OK (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/685,716

(22) Filed: Mar. 13, 2007

Related U.S. Application Data
(60) Provisional application No. 60/782,192, filed on Mar. 14, 2006.

(51) Int. Cl. A61B 5/00

(52) U.S. Cl. 600/323; 600/344

(57) ABSTRACT

A pulse oximeter sleeve of elastic and/or form-fitting material for positioning light-emitting diodes and a detector securely with respect to a human's foot or hand.

### PRIORITY CLAIM

The present application claims priority benefit under 35 U.S.C. § 119(e) to U.S. Provisional Patent Application Ser. No. 60/782,192, filed Mar. 14, 2006, by the same title. The present application incorporates the foregoing disclosures herein by reference.

### CROSS-REFERENCE TO RELATED APPLICATIONS

The present application is related to U.S. Pat. No. 6,470,199, issued on Oct. 22, 2002, titled “Elastic Sock for Positioning an Optical Probe.” The present application incorporates the foregoing disclosures herein by reference.

### BACKGROUND OF THE DISCLOSURE

1. Field of the Disclosure

This present disclosure relates to the field of pulse oximetry, and more particularly attachment devices that are used to secure a pulse oximeter optical probe element about a human foot or hand. In an embodiment, the attachment devices advantageously reduce a likelihood that the pulse oximeter will be dislodged from the foot or hand.

2. Description of the Related Art

The technology of pulse oximetry is well known in the art. Caregivers can use pulse oximeters to measure among other things the amount of oxygen in a patient's blood. A common type of pulse oximeter uses light-emitting diodes to direct light of predetermined wavelengths through a translucent part of the patient's body such as a fingertip. The pulse oximeter also has a photodetector that receives the emitted light so that the amount of light that was absorbed by the patient's blood can be measured. That measurement is then used to determine among other things the amount of oxygen in the patient's blood. Exemplary pulse oximeters are commercially available from Masimo Corporation of Irvine, Calif. Moreover, exemplary pulse oximeters are disclosed in at least U.S. Pat. Nos. 5,490,505; 5,482,036; 5,632,272; 6,584,336 and 6,770,028, each of which is incorporated herein.

Reusable probes are typically attached to a patient's fingertip with a clip that attaches over the end of the fingertip such that the light-emitting diodes are positioned over the fingernail. The disadvantage of the clip is that it may to fall off or to be dislodged from the patient's fingertip.

Further, clips are sometimes disadvantageous to use in connection with infants. For example, infant fingertips are small, making it difficult to obtain an accurate reading and making the clip more likely to be dislodged. Thus, pulse oximeters have often been attached to an infant's foot with an disposable adhesive strip that wraps around the foot such that the light-emitting diodes are positioned over the thinner part of the infant's foot near the toes. One disadvantage of the adhesive strip is that it may tend to fall off the infant's foot or to be dislodged as the infant's foot moves about. Another disadvantage is that the adhesive strip may contain latex to which some infants are allergic. Moreover, the adhesive may harbor bacteria and potential pathogens as a result of the skin underneath the adhesive obtaining and retaining moisture.

### SUMMARY OF THE DISCLOSURE

The present disclosure addresses and solves the problems of the prior art by providing a sleeve of elastic and form-fitting material that substantially fixes the light-emitting diodes and photodetector in place with respect to one another and to the tissue to which the sleeve is attached. In one embodiment the sleeve is similar to a sock and is used to position the light-emitting diodes and photodetector in place near the toes on an infant's foot. The sleeve is open at the toes. At least some open toes advantageously provide visibility of the toes in the event that the sleeve is too tight. For example, tightness may cause the infant's toes to become blue.

In addition, the sleeve may advantageously extend past the ankle to assist in ensuring that it remains securely in place. The light-emitting diodes, photodetector, and in the case of wired communications, the accompanying wiring are sewn into the fabric of the sleeve or otherwise attached to the sleeve in the appropriate position. In an embodiment, the sleeve may include modular housings adapted to snap fit electronic elements of a probe into place. Exemplary snap fit or other matable housings are disclosed in, for example, U.S. Pat. Nos. 6,920,345; 6,343,244; 6,735,459; 6,519,487; 6,011,986, each of which are incorporated herein.

In another embodiment of the disclosure, the sleeve is similar to a glove wherein the sleeve covers substantially the entire hand and some or the entirety of at least one finger. The remaining fingers and thumb may be free to provide added comfort to the individual wearing the sleeve and to ensure visual confirmation of good perfusion. The portion of the sleeve covering one finger has the light-emitting diodes and the photodetector positioned near the fingertip.

In some or all of the foregoing embodiments, the pulse oximeter is capable of giving a continuous and more accurate reading because it is held in place. Further, the sleeve may advantageously eliminate interference from outside light sources that can affect the pulse oximeter reading. Additionally, the sleeve may advantageously warm the skin of the patient to further increase the accuracy of the reading.

For purposes of summarizing the disclosure, certain aspects, advantages and novel features of the disclosure have been described herein. Of course, it is to be understood that not necessarily all such aspects, advantages or features will be embodied in any particular embodiment of the disclosure.

### BRIEF DESCRIPTION OF THE DRAWINGS

The following drawings and the associated descriptions are provided to illustrate embodiments of the present disclosure and do not limit the scope of the claims.

FIG. 1 is a perspective view illustrating one embodiment of the prior art used in conjunction with a human hand;

FIG. 2 is a perspective view illustrating another embodiment of the prior art used in conjunction with a human foot;

FIG. 3 is a perspective view of a sock used in conjunction with a human foot; and

FIG. 4 is a perspective view of a glove used in conjunction with a human hand.

### DETAILED DESCRIPTION OF THE PREFERRED EMBODIMENT

To facilitate a more complete understanding of the disclosure, the Detailed Description describes the disclosure with reference to the drawings, wherein like reference numbers are referenced with like numerals throughout.

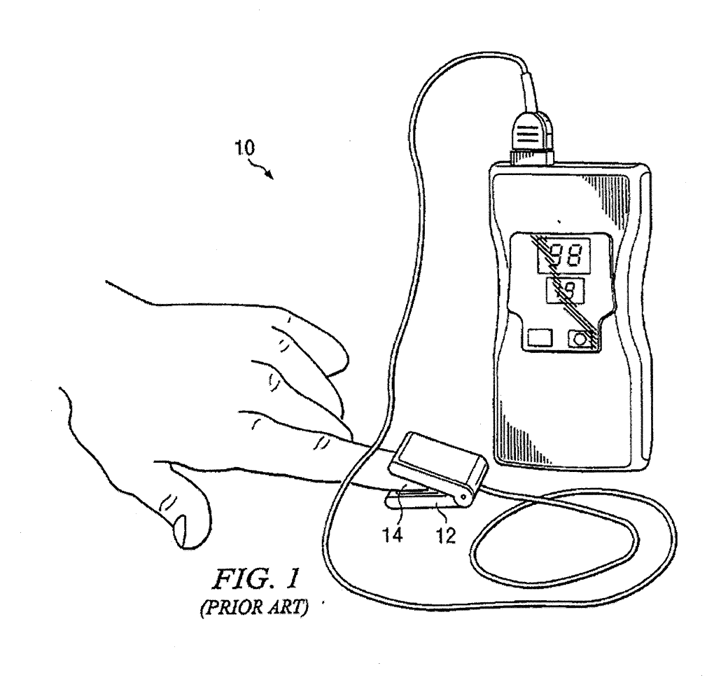

Referring to the drawings, and in particular to FIG. 1, there is shown a perspective view of a conventional pulse oximeter 10, wherein the pulse oximeter 10 comprises a clip 12 that is attached to an individual's fingertip 14.

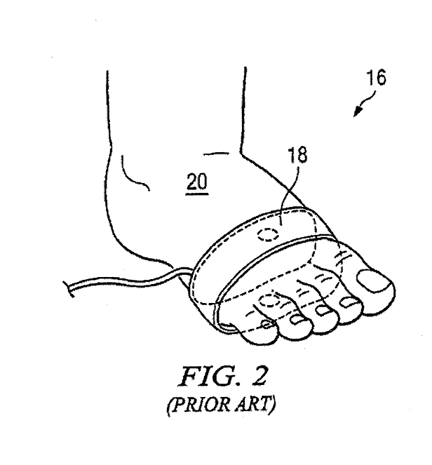

FIG. 2 is a perspective view of another conventional pulse oximeter 16, wherein an adhesive strip 18 is used to position the pulse oximeter 16 on an infant's foot 20.

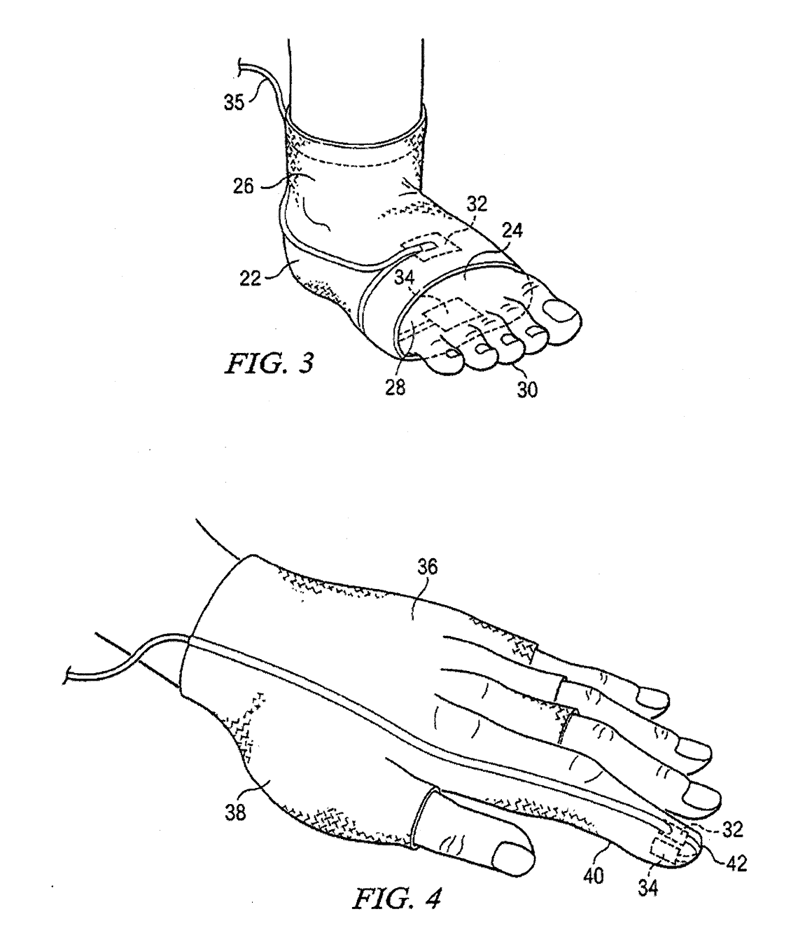

In FIG. 3 there is shown a perspective view of one embodiment of the pulse oximeter sleeve 22 of the disclosure wherein the sleeve 22 is positioned about an infant's foot 24 and ankle 26 and has an open end 28 through which the infant's toes 30 extend. In this embodiment the light-emitting diodes 32 are located on top of the infant's foot 24 near the toes 30, and the photodetector 34 is positioned directly below so that the photodetector 34 can receive the light emitted by the light-emitting diodes 32 and not absorbed by the blood within the infant's foot 24 as the light passes therethrough. Also shown is wiring 35 sewn into the sleeve 22 for transmitting information generated by the light emitting diodes 32 and the photodetector 34 to a medical monitor. An artisan will recognize from the disclosure herein that the optical probe may advantageously be reflective, transmissive, disposable, reusable, some combination of the foregoing, or the like.

In FIG. 4 there is shown a perspective view of another embodiment of the disclosure wherein the sleeve comprises a glove 36 that covers substantially the entire hand 38 of an individual and the entirety of at least one finger 40. The portion of the glove 36 that covers the finger 40 has positioned thereon near the fingertip 42 the light-emitting diodes 32 on one side of the finger 40 and the photodetector 34 on the opposite side.

Although preferred embodiments of the disclosure have been illustrated in the accompanying Drawings and described in the foregoing Detailed Description, it will be understood that the disclosure is not limited to the embodiments disclosed, but is capable of numerous rearrangements, modifications, and substitutions of parts and elements without departing from the spirit of the disclosure. For example, a more than two wavelength optical probe may be positioned using the disclosed sleeve embodiments; monitors monitoring different parameters may be employed, tightness may be advantageously be controlled through sleeve size, adjustments such as Velcro straps, or the like. Moreover, one or more digits may extend out of the sleeve to ensure visual confirmation of blood perfusion through tissue. Additionally, other combinations, omissions, substitutions and modifications will be apparent to the skilled artisan in view of the disclosure herein. Accordingly, the present invention is not intended to be limited by the reaction of the preferred embodiments, but is to be defined by reference to the appended claims.

Additionally, all publications, patents, and patent applications mentioned in this specification are herein incorporated by reference to the same extent as if each individual publication, patent, or patent application was specifically and individually indicated to be incorporated by reference.

What is claimed is:

1. A pulse oximeter system for positioning elements of an optical probe capable of measuring physiological parameter of a patient, the system comprising:
a substantially tubular sleeve comprising an elastic and form-fitting material having two open ends for secure positioning about a human foot, ankle or hand so that one or more digits are not enclosed;
at least one light-emitting diode positioned by said sleeve to emit light into said foot, ankle or hand; and
a detector positioned by said sleeve to detect said light after attenuation by tissue of said foot, ankle or hand.

2. The system of claim 1, further comprising means responsive to the detector for determining said physiological parameter.

3. The system of claim 1, wherein said digits comprise toes.

4. The system of claim 3, wherein one of said open ends does not enclose two or more of said toes.

5. The system of claim 3, wherein one of said open ends does not enclose all of said toes.

6. The system of claim 1, wherein said digits comprise fingers.

7. The system of claim 6, wherein one of said open ends does not enclose two or more of said fingers.

8. The system of claim 6, wherein one of said open ends does not enclose all of said fingers.

9. A pulse oximeter glove for positioning elements of an optical probe capable of measuring physiological parameter of a patient, the glove comprising an elastic and form-fitting material capable of positioning at least one light-emitting diode and a detector proximate a finger to allow said detector to detect light after attenuation by tissue of a said finger, wherein said glove does not enclose at least one finger.

10. The glove of claim 9, wherein said glove does not enclose two or more fingers.

11. The glove of claim 10, wherein said glove does not enclose all of the fingers of a hand.

12. A pulse oximeter sock for positioning elements of an optical probe capable of measuring physiological parameter of a patient, the sock comprising an elastic and form-fitting material capable of positioning at least one light-emitting diode and a detector proximate a toe to allow said detector to detect light after attenuation by tissue of a said toe, wherein said sock does not enclose at least one toe.

13. The glove of claim 12, wherein said sock does not enclose two or more toes.

14. The glove of claim 13, wherein said sock does not enclose all of the toes of a foot.
